(19) United States
(12) Patent Application Publication
(10) Pub. No.: US 2004/0117003 A1
(43) Pub. Date: Jun. 17, 2004

(54) Minimally invasive treatment system for aortic aneurysms

(75) Inventors: Kenneth Ouriel, Pepper Pike, OH (US); Harry B. Goodson, Fremont, CA (US); Lisa K. Jordan, Philadelphia, PA (US)

Correspondence Address: TAROLLI, SUNDHEIM, COVELL & TUMMINO L.L.P., SUITE 1111, 526 SUPERIOR AVENUE, CLEVELAND, OH, 44114-1400, US

(73) Assignee: The Cleveland Clinic Foundation

(21) Appl. No.: 10/446,472

(22) Filed: May 28, 2003

Related U.S. Application Data
(60) Provisional application No. 60/383,524, filed on May 28, 2002.

(51) Int. Cl. A61F 2/06

(52) U.S. Cl. 623/1.35; 623/1.16; 623/1.3

(57) ABSTRACT

An endoluminal prosthesis (10) comprises a radially expandable tubular segment (12) having a first end (32), a second end (34), a lumen interconnecting the first end (32) and the second end (34). A connection portion (52) defines an opening in the tubular segment (12) in fluid communication with the lumen. The connection portion (52) includes a converging portion (54), an annular diverging portion (56) and an annular neck portion (58) interconnecting the converging portion (52) and the diverging portion (56).

### FIELD OF THE INVENTION

The present invention relates to vascular surgical devices. More specifically, it relates to endoluminal prostheses for the repair of vascular defects, such as aortic aneurysms.

### BACKGROUND OF INVENTION

Standard treatment for aortic aneurismal disease involves replacement of the diseased portion of the aorta with a synthetic graft via an open surgical approach. Surgery for abdominal aortic aneurysm (AAA) repair involves a midline abdominal or retroperitoneal incision to gain access, with significant organ and bowel dislocation and manipulation necessary to reach the aorta along the spine. For thoracic aortic aneurysm (TAA) repair, an approach is generally made from the patient's left chest, often necessitating left lung and kidney displacement and possibly involving the removal of one or more ribs to gain adequate access. In either case, the affected portion of aorta is opened, debris removed, and bypassed with a prosthetic graft. The repair is generally viewed as durable and is the “gold standard” of treatment.

The treatment of aortic aneurysms is changing due to the innovation of minimally invasive therapy. Endovascular treatment for aortic aneurysms, in contrast to standard open surgical repair, requires only small, bilateral groin incisions to access the external iliac or common femoral arteries. This offers the promise of reduced operative time, associated risk, recovery time, and blood loss, as well as completion without the use of general anesthetic.

There are two devices currently available in the United States for such treatment, the Ancure Endograft System and the AneuRx Stent Graft System, marketed by Guidant (Menlo Park, Calif.) and Medtronic AVE (Santa Rosa, Calif.), respectively. Numerous other devices are available overseas, and in FDA-approved investigational device exemption (IDE) trials in the U.S. As summarized by the extensive EUROSTAR Registry, endovascular treatment can provide lower acute morbidity and mortality compared to an open surgical approach, allowing for reduced ICU time as well as earlier ambulation and discharge.

In general, an endovascular stent graft consists of a stent (frame) component and a graft (fabric) component. A device for AAA treatment may be tubular (aorto-aortic or aorto mono-iliac) or bifurcated (aorto bi-iliac). The stent graft may be modular (i.e., with the body and limbs deployed separately, having the ability to be adjusted in vivo with add-on pieces) or unibody (i.e., one piece) in design. For TAA treatment, devices are tubular (aorto-aortic). Stent grafts may be self-expanding (i.e., it expands spontaneously when released from its delivery system), balloon expandable (i.e., requiring adjunct internal pressure to expand it), or they may be a combination of these two.

The metallic stent frame component is intended to support the device, maintain its physical configuration, and provide an opening force upon deployment. The stent structure is often integral in maintaining the position of the device within the vasculature and providing for its sealing to the vessel. The stent component may be formed of stainless steel, other similar metal, or an alloy, such as NITINOL.

The polymeric graft component is the artificial blood vessel (conduit), designed to provide a path through which blood is re-directed, thereby excluding the aneurysmal segment of the vessel from blood pressure and flow. This reduces the propensity of the aneurismal segment to rupture. The graft component is usually formed of a woven or knitted polyester (PET) or expanded polytetrafluoroethylene (ePTFE). For delivery into and deployment within the vasculature, the stent graft is loaded into a delivery system, such as a catheter-based device that can be guided to the desired site and that can then release the stent graft into position under fluoroscopic guidance.

An endovascular stent graft that is designed for permanent implantation inside the human body must be able to withstand the environment in which it will reside. It is assumed that the stent graft needs to maintain its full functionality over time, as the disease process does not “get better” by placement of the device. Therefore, theoretically, a stent graft must indefinitely maintain its physical, chemical, and mechanical properties while being subjected to the environmental factors of the human aorta. The simulation of the aortic environment is in itself a challenging endeavor, and one not completely understood.

No durability test can simulate an infinite time period, so in order to provide an attainable goal the FDA requires demonstration of a ten-year service life for cardiovascular implants. The predictable, cyclic displacements within the body to which the device may be exposed include the beating of the heart and the expansion and contraction of the lungs. A proposed device must withstand approximately 420,000,000 cardiac cycles and 63,000,000 respiratory cycles, taking the average human heart rate as 80 beats per minute and the average respiratory rate as 12 breaths per minute. Study of human anatomy and physiology leads to the conclusion that cardiac cycles should impart radial, torsional, and, to a lesser extent, axial, loading on the region of the aorta where an endovascular repair would be completed, while respiratory cycles should impart axial, bending, and possibly torsional loading.

### SUMMARY OF THE INVENTION

The present invention relates to an endoluminal prosthesis that comprises a radially expandable tubular segment having a first end, a second end, a lumen interconnecting the first end and the second end, and a connection portion defining an opening in the tubular segment in fluid communication with the lumen. The connection portion includes a converging portion, an annular diverging portion, and an annular neck portion interconnecting the converging portion and the diverging portion.

In accordance with another aspect of the present invention, the endoluminal prosthesis can comprise a second radially expandable tubular segment. The second segment can have a first end, a second end, a lumen interconnecting the first end and the second end, and a second connection portion defining an opening in a mid-portion of the second tubular segment between the first end and the second end of the second segment. The opening in the mid-portion can be in fluid communication with the lumen of the second segment. The second connection portion can be capable of joining with the first connection portion in situ to form a mechanical junction that allows fluid flow between the first segment and the second segment.

In accordance with yet another aspect of the present invention, the endoluminal prosthesis can be used to treat an infrarenal abdominal aortic aneurysm or a suprarenal abdominal aortic aneurysm. Where the endoluminal prosthesis is used to treat a suprarenal abdominal aortic aneurysm, the endoluminal prosthesis can include a radially expandable tubular trunk segment having a first end, a second end, a lumen interconnecting the first end and the second end, and at least two connection portions defining openings in a mid-portion of the trunk segment between the first end and the second end of the trunk segment. The openings in the mid-portion can be in fluid communication with the lumen of the trunk segment. At least one of the connection portions can comprise an annular converging portion, an annular diverging portion and an annular neck portion interconnecting the converging portion and the diverging portion.

The endoluminal prosthesis used to treat a suprarenal abdominal aortic aneurysm can also comprise a radially expandable tubular branch segment. The branch segment can have a first end, a second end, a lumen interconnecting the first end and the second end, and a second connection portion. The second connection portion being capable of joining with at least one of the connection portions of the trunk segment in situ to form a mechanical junction that allows fluid flow between the trunk segment and the branch segment.

The present invention also provides a method of treating an aortic aneurysm. According to the inventive method, a first radially expandable tubular segment can be deployed. The first segment can have a first end, a second end, a lumen interconnecting the first end and the second end, and a first connection portion defining an opening in a mid-portion of the first segment between the first end and the second end. The first connection portion can include a converging portion, an annular diverging portion and an annular neck portion interconnecting the converging portion and the diverging portion. A second radially expandable tubular segment can also be deployed. The second segment can include a distal end, a proximal end, a lumen interconnecting the distal end and the proximal end, and a second connection portion defining an opening in the proximal end in fluid communication with the lumen. The second connection portion can include a converging portion, an annular diverging portion and an annular neck portion interconnecting the converging portion and the diverging portion. The second connection portion and the first connection portion can form an end-to-side junction, which allows fluid flow between the first segment and the second segment.

A further aspect of the present invention relates to an endovascular prosthesis that comprises a radially expandable tubular graft layer having a first end, a second end, and a lumen extending between the first end and the second end. The first end can include a plurality of substantially radially oriented hooks that extend from the graft layer to provide a fluid tight seal between graft layer of the first end and a wall of the vasculature. The hooks can enter the wall of the vasculature in a rotational manner to draw the first end into close apposition to the wall. The hooks can extend in a substantially coplanar configuration that is essentially perpendicular to blood flow through the endovascular prosthesis. The hooks can be deployed in an essentially geometric plane that is essentially perpendicular to the blood flow within the vasculature.

In accordance with another aspect of the present invention the endovascular prosthesis can comprise a radially expandable tubular graft layer having a first end, a second end, and a lumen extending along an axis between the first end and the second end. The first end can include an anchoring means for substantially inhibiting axially motion of the endovascular prosthesis relative to the vasculature and a sealing means to provide a fluid tight seal between graft layer of the first end and a wall of the vasculature. The anchoring means and sealing means can be separate from one another.

In a further aspect of the present invention, the sealing means can comprise a plurality of substantially radially oriented hooks. The hooks can be curved to enter a wall of a vasculature upon axial rotation of the endovascular prosthesis and draw the first end into close apposition to the wall so as to form a fluid tight seal between the graft layer of the first end and the wall of the vasculature. The anchoring means can include a second plurality of hooks, which can penetrate the of wall the vasculature. The hooks of the anchoring means can be deployed at an angle less than 90 degrees with the direction of blood flow through the endovascular prosthesis.

Another aspect of the present invention provides a method of deploying the endovascular prosthesis within a vasculature. According to the inventive method, an endovascular prosthesis can be provided that includes a radially expandable tubular graft layer having a first end, a second end, a lumen extending between the first end and the second end. A plurality of substantially radially oriented hooks can extend from the first end. The hooks can be curved to enter a wall of the vasculature upon axial rotation of the endovascular prosthesis. The substantially radially oriented hooks of the first end can be rotationally embedded into the wall of the vasculature to achieve a fluid-tight seal.

A further aspect of the present invention relates to a method of forming a fluid tight seal between an endovascular prosthesis and a wall of a vasculature. According to the inventive method, an endovascular prosthesis can be provided that includes a radially expandable tubular graft layer having a first end, a second end, a lumen extending between the first end and the second end. A plurality of substantially radially oriented hooks can extend from the first end. The hooks can be curved to enter a wall of the vasculature upon axial rotation of the endovascular prosthesis. The substantially radially oriented hooks of the first end can be rotationally embedded into the wall of the vasculature.

### BRIEF DESCRIPTION OF THE DRAWINGS

The foregoing and other features of the present invention will become apparent to those skilled in the art to which the present invention relates upon reading the following description with references to the accompanying drawings, in which:

FIG. 1 is a perspective view of an endoluminal prosthesis in accordance with an aspect of the present invention;

FIG. 2 is a perspective view of the aortic module of the endoluminal prosthesis of FIG. 1;

FIG. 3 is a perspective view of the bi-iliac module of the endoluminal prosthesis of FIG. 1 in accordance with an aspect of the invention;

FIG. 4 is a perspective view of the bi-iliac module of the endoluminal prosthesis of FIG. 1 in accordance with another aspect of the present invention;

FIGS. 5a-5d Illustrate a method of deploying the endoluminal prosthesis of FIG. 1 to treat an abdominal aortic aneurysm;

FIG. 6 is a perspective view of the proximal sealing collar module of an endoluminal prosthesis in accordance with another aspect of the present invention;

FIG. 7 is a perspective view of an aortic module in accordance with another aspect of the present invention;

FIG. 8 is a perspective view of the proximal sealing collar of FIG. 6, the aortic main body module of FIG. 7, and the bi-iliac module of FIG. 3 implanted in an abdominal aortic aneurysm;

FIG. 9 is a perspective view of a suprarenal module of an endoluminal prosthesis in accordance with another aspect of the invention;

FIG. 10 is a perspective view of a branch module of an endoluminal prosthesis in accordance with another aspect of the invention;

FIG. 11 is a perspective view of the suprarenal module of FIG. 9, the branch modules of FIG. 10, and aortic modules of FIG. 2 implanted in a suprarenal aortic aneurysm;

FIG. 12 is a perspective view of another suprarenal module of an endoluminal prosthesis in accordance with another aspect of the present invention; and

FIG. 13 is a perspective view of the suprarenal module of FIG. 12 and branch modules of FIG. 10 implanted in a suprarenal aortic aneurysm.

### DESCRIPTION OF THE PREFERRED EMBODIMENTS

The present invention relates to an endoluminal prosthesis that can be used to treat a vascular disorder. The endoluminal prosthesis includes at least two segments that can be joined together in situ (as well as in vivo) to form the endoluminal prosthesis. At least two of the segments include connection portions for joining the segments. Each connection portion includes a converging portion, a diverging portion, a neck portion that interconnects the converging portion and the diverging portion. The connection portions can engage one another to form a mechanical junction. The endoluminal prosthesis formed by joining the segments can be used to treat an aortic aneurysm that extends to or into branching arteries of the aorta.

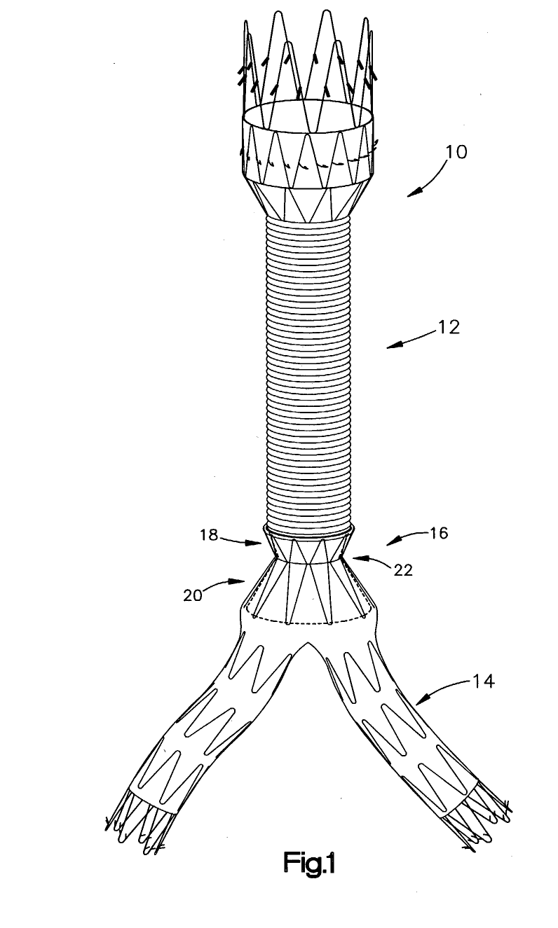

FIGS. 1-4 illustrate a perspective view of an endoluminal prosthesis 10 in accordance with one aspect of the present invention that can be used to treat an abdominal aortic aneurysm that extends from a portion of the aorta caudal the renal arteries to the aorta-iliac junction (i.e., infrarenal abdominal aortic aneurysm). The endoluminal prosthesis 10 has a modular design that includes an aortic module 12 and a bi-iliac module 14. The aortic module 12 and the bi-iliac module 14 can connect at a junction 16. The junction 16 can have an essentially hourglass shape with a converging portion 18, a diverging portion 20, and a neck portion 22 that interconnects the converging portion 18 and the diverging portion 20.

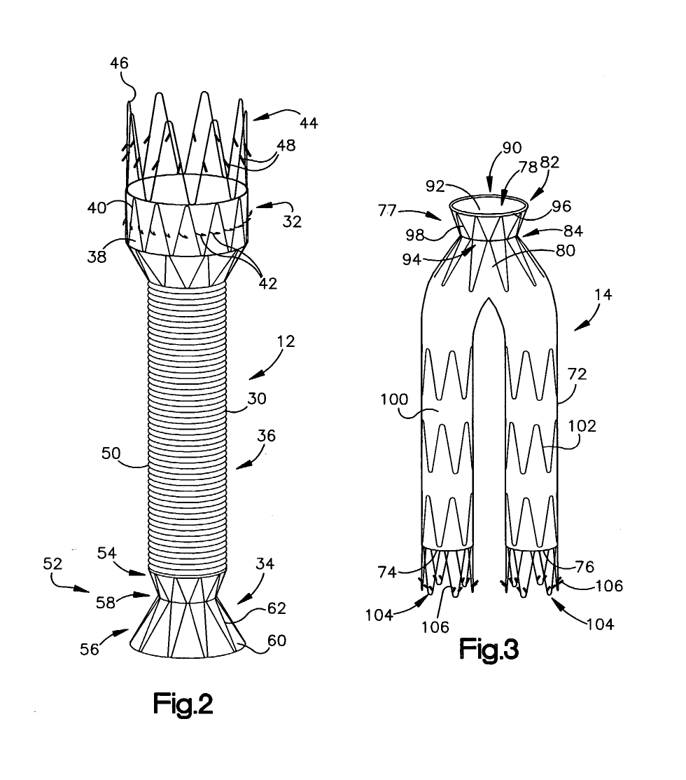

FIG. 2 is a perspective view of the aortic module 12. The aortic module 12 comprises a flexible substantially unsupported, and highly conformable tubular structure 30. The flexible, substantially unsupported tubular structure 30 of the aortic module 12 readily conforms to the arterial system acutely as well as accommodates without significant resistance future re-modeling of the arteries, which may occur due to factors such as sac shrinkage and/or arterial disease progression.

The aortic module 12 includes a proximal end 32, a distal end 34, and a main body portion 36 that interconnects the proximal end 32 and the distal end 34. The proximal end 32 has a substantially frustoconical shape that is radially supported at least a portion of the length of the proximal end 32. The proximal end 32 provides a fluid-tight seal between the proximal end 32 and the aorta in order to create a conduit for blood flow, with full, leak-free exclusion of the aortic aneurysm. The distal end 34 serves as a mechanical junction (i.e., a docking zone) for the bi-iliac module 14.

The proximal end 32 includes at least one graft layer 38 and a means 40 for radially supporting the graft layer 38. The graft layer 38 comprises a fabric having sufficient strength to withstand the surgical implantation of the aortic module 12 and to withstand the blood pressure and other biomechanical forces that are exerted on the proximal end 32. The fabric can be formed by weaving or extruding a biocompatible material. Examples of biocompatible materials, which can be weaved or extruded to form the graft layer, are polyethylene, polypropylene, polyurethane, polyglycolic acid, polyesters, polyamides, polyfluorocarbons, copolymers thereof, and mixtures thereof. Preferred biocompatible materials, which can be used to form the graft layer, are polyesters, such as DACRON and MYLAR, and polyfluorocarbons, such as polytetrafluoroethylene and expanded polytetrafluoroethylene (ePTFE).

The biocompatible fabric can be an expanded polytetrafluoroethylene fabric (ePTFE) that is formed, in a manner not shown, by extruding a polytetrafluoroethylene-lubricant mixture through a ram extruder into a tubular-shaped extrudate and longitudinally expanding the tubular extrudate to yield a uniaxially oriented fibril microstructure in which substantially all of the fibrils in the expanded polytetrafluoroethylene (ePTFE) microstructure are oriented parallel to one another in the axis of longitudinal expansion.

The means 40 for radially supporting the graft layer can comprise a stent 40. The stent(s) 40 can have a construction similar to any radially expandable stent well-known in the art, which is suitable for vascular implantation. For example, the stent 40 can include a plurality of axially aligned radially expandable stents. Each stent 40 can include an annular support beam, which has a generally sinusoidal shape. The wavelength of each of the support beams can be identical or essentially identical to the wavelength of the adjacent axially aligned support beams.

The stent 40 can be formed of a metal that has super-elastic properties. Preferred metals include nickel-titanium alloys. An example of a nickel-titanium alloy is NITINOL. Nickel-titanium alloys are preferred as metals for the stent 40 because of their ability to withstand a significant amount of bending and flexing and yet return to their original shape without deformation. Nickel-titanium alloys are also characterized by their ability to be transformed from one shape with an austenitic crystal structure to another shape with a stress induced martensitic crystal structure at certain temperatures, and to return elastically to the one shape with the austenitic crystal structure when the stress is released. These alternating crystal structures provide nickel-titanium alloys with their super-elastic properties. Examples of other metals that have super-elastic properties are cobalt-chrome alloys (e.g., ELGILOY) and platinum-tungsten alloys.

Other materials that can be used to form the stent 40 are metals, such as stainless steel, and polymeric materials, such as nylon, and engineering plastics, such as thermotropic liquid crystal polymers. Thermotropic liquid crystal polymers are high molecular weight materials that can exist in a so-called “liquid crystalline state” where the material has some of the properties of a liquid (in that it can flow) but retains the long range molecular order of a crystal. Thermotropic liquid crystal polymers may be prepared from monomers such as p,p′-dihydroxy-polynuclear-aromatics or dicarboxy-polynuclear aromatics.

The stent 40 can be fixedly attached to the inner surface or outer surface of the graft layer 38 or integrated into the graft layer 38. The stent 40 can be attached to the inner surface or outer surface of the graft layer 38 by mechanical means. An example of a mechanical means is a suture that is used to sew the stent 40 to the inner surface or outer surface of the graft layer 38. Alternatively, the stent 40 can be attached to the inner surface or outer surface of the graft layer 38 by a polymer adhesive layer (not shown). Examples of polymer adhesive layers include a silicone based layer and polyurethane based layer.

In yet another configuration (not shown), the proximal end 32 can include two graft layers that are coaxially aligned and fixedly attached to one another by a polymer adhesive layer. The two graft layers can comprise the same fabric or a different fabric. A means for radially supporting a graft layer can be fixedly attached to the inner layer or outer layer of one of the graft layers.

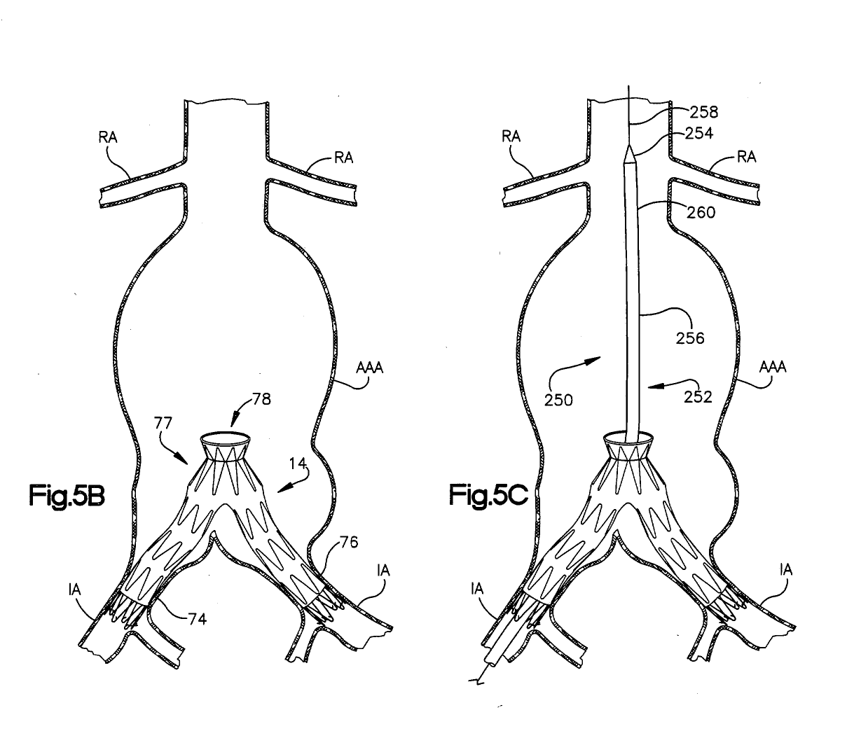

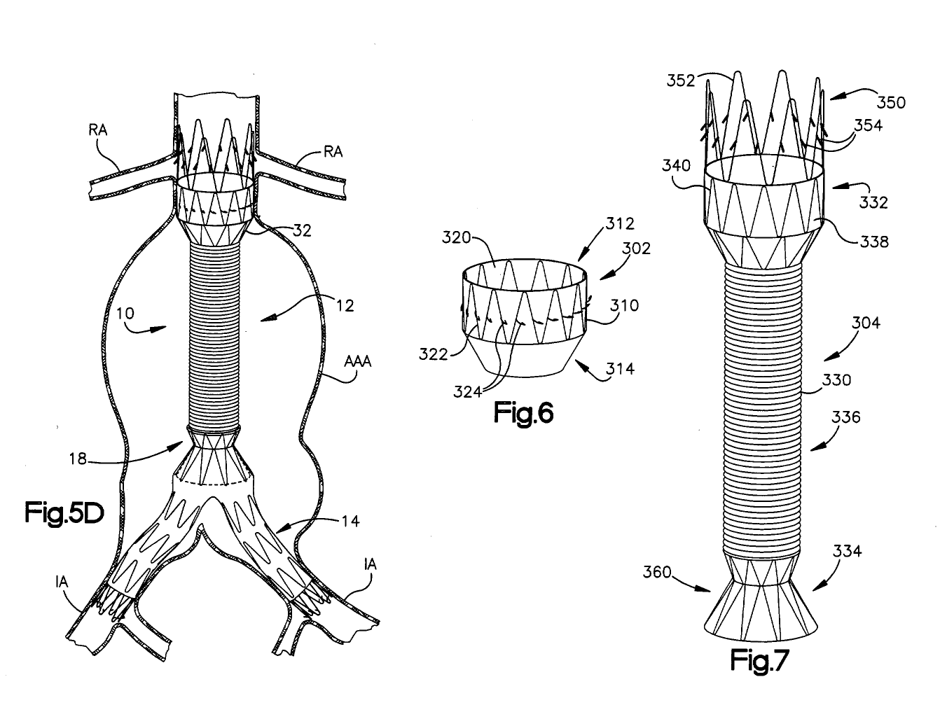

The proximal end 32 of the aortic module also includes a plurality of substantially radially oriented hooks 42 for ensuring sealing of the proximal end 32 of the aortic module 12 within the aorta. Preferably, the hooks 42 are curved and extend in an outward direction from the proximal end 32 of the aortic module 12 such that when the proximal end 32 is rotated within the aorta, the hooks are deployed, i.e., rotationally embedded, into the wall of aorta, as shown in FIG. 5D. Deployment of the radially oriented hooks 42 into the aorta mimics a surgeon's suture and provides secure apposition of the proximal end 32 of the aortic module 12 to the aorta. The hooks 42 can extend in a substantially coplanar configuration that is essentially perpendicular to the axially extending proximal end and to the blood flow through the proximal end of the aortic module. The hooks 42 can be deployed in an essentially geometric plane, which is substantially perpendicular to the blood flow within the aorta.

An anchoring means 44 can extend from the proximal end 32 of the aortic module 12. The anchoring means 44 can comprise a radially expandable bare stent 46. By “bare stent” it is meant that the stent is not covered with a graft layer or fabric that would inhibit radial flow of fluid through the stent. The bare stent 46 can be substantially tubular and can have a construction similar to any vascular stent known in the art.

The bare stent 46 can include axially aligned barbs 48 (or hooks) that extend outwardly from the bare stent 46 and at an angle less 90 degrees with the direction of blood flow through the proximal end 32. When the bare stent 46 is radially expanded, the barbs 48 engage the wall of the aorta and prevent axial migration of the aortic module 12 within the aorta.

The main body portion 36 comprises a highly flexible unsupported tubular graft material. By “unsupported”, it is meant that the main body portion 36 does not include a support means, such as a stent, to provide radial support. Preferably, the main body portion 36 has corrugated construction formed from radially crimped fabric. The radially crimped fabric includes at least one graft layer. The fabric used to form the one graft layer can be similar to the fabric used to form proximal end 32. Other graft fabrics well known in the art can also be used. The radially crimped fabric can also include additional layers. These additional layers can include other grafts layers and polymer adhesive layers.

The distal end 34 of the aortic module 12 can include a connection portion 52 that defines an opening (not shown) in the distal end 34. The connection portion 52 can have an annular converging portion 54, an annular diverging portion 56, and an annular neck portion 58 that interconnects the converging portion 54 and the diverging portion 56. The converging portion 54 and the diverging portion 56 can taper radially inward to the neck portion 58. The converging portion 54 and diverging portion 56 can both have an essentially frustoconical shape, which provides the distal end 34 with an essentially hourglass configuration. As shown in FIG. 5D, the hourglass configuration allows the distal end 34 of the aortic module 12 to be connected to the bi-iliac module 14 (Fid. 3) in-situ and form the junction 16 that is similar to an end-to-side surgical anastomosis.

The hourglass distal end 34 can be formed from at least one graft layer 60 and a means 62 for radially supporting the graft layer (e.g., annular stent). The construction of distal end 34 can be similar to the construction of the proximal end 32 of the aortic module 12.

FIG. 3 is a perspective view of the one-piece bi-iliac module 14. As shown in FIGS. 5B and 5C, the one-piece bi-iliac module 14 bridges the aortic bifurcation, extending between an iliac or femoral artery on one side and an iliac or femoral artery on the other.

Referring again to FIG. 3, the bi-iliac module 14 comprises a flexible hollow tubular segment 72 that defines a main lumen (not shown) between an open first end 74 and an open second end 76. The first end 74 and the second end 76 are in fluid communication with each other by the main lumen of the segment 72.

The bi-iliac module 14 further includes a connection portion 77 that defines a side opening 78 in the segment between the first end 74 and second end 76. The side opening 78 is in fluid communication with the first end 74 and the second end 76, such that fluid flow will be allowed into the side opening of the tubular segment 72 and out of the openings at the first and second ends, 74 and 76.

The side opening 78 can be located about halfway between the two ends 74 and 76 of the segment. Preferably, the tubular segment 72 has an inverted U-shape and the side opening 78 is in a mid-portion of the tubular segment 72 at about the apex of the inverted U-shape.

The connection portion 77 can include an annular converging portion 80, an annular diverging portion 82, and an annular neck portion 84 that interconnects the converging portion 80 and the diverging portion 82. The converging portion 80 and the diverging portion 82 can taper radially inward to the neck portion 84. The diverging portion 82 can be essentially frustoconical (i.e., funnel shaped) in configuration and perpendicularly offset from the lumen of the bi-iliac module 14 to provide the connection portion 77 with an essentially hourglass configuration. As shown in FIG. 5D, the hourglass connection portion 77 of the bi-iliac module 14 allows the distal end 34 of the aortic module 12 to be connected to the bi-iliac module 14 with a mechanical locking fit.

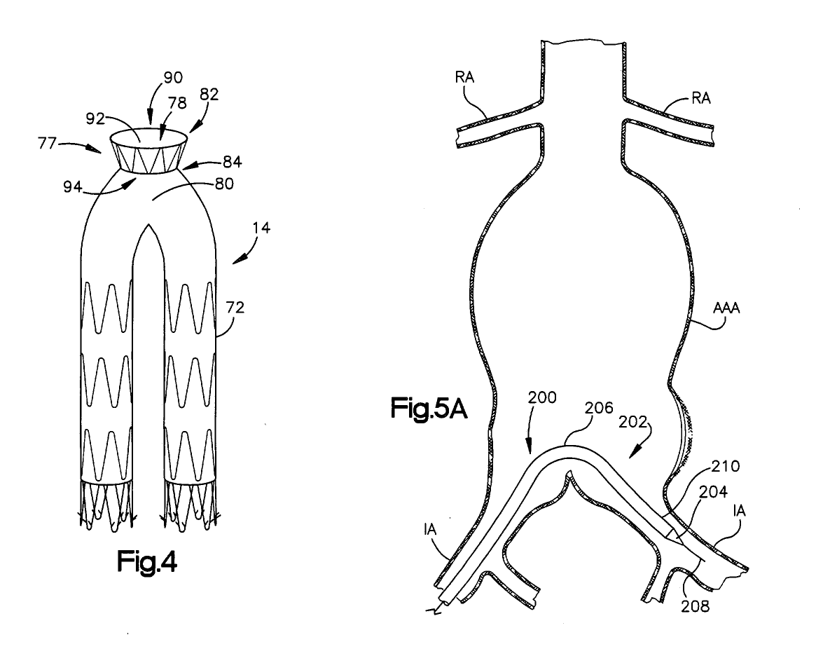

The diverging portion 82 has a proximal end 90 that defines a first opening 92 and a distal end 94 that defines a second opening (not shown) substantially smaller that the first opening 92. The second opening 94 can be supported in open configuration by a support member, such as a radially expandable stent. The first opening 92 can be supported in an open configuration by a resilient ring 96 (e.g., NITINOL or a resilient polymer) that is incorporated in the proximal end 90 of the diverging portion 82 at or near the first opening 92 and a tapered stent 98 that extends along at least a portion of the inwardly directed diverging portion 82. The second opening can be supported in substantially open configuration by a support member, such as a tapered stent. Optionally, as shown in FIG. 4, the second opening can be provided without the support member and the first opening can be provided without the resilient ring.

The bi-iliac module 14 can be formed from at least one graft layer 100 and a means 102 for radially supporting the graft layer 100. The graft layer 100 can comprise a fabric having sufficient strength to withstand the surgical implantation of the bi-iliac module 14 and to withstand the blood pressure and other biomechanical forces that are exerted on the bi-iliac module.

The means 102 for radially supporting the graft layer can comprise a stent 102 that provides lumen patency to the bi-iliac module 14 in the tortuous iliac and femoral arteries. The stent(s) 102 can have a construction similar to any radially expandable stent well-known in the art, which is suitable for vascular implantation. The stent 102 can be fixedly attached to the inner surface or outer surface of the graft layer 100 or integrated into the graft layer 100. The stent 102 can be attached to the inner surface or outer surface of the graft layer 100 by mechanical means. Alternatively, the stent 102 can be attached to the inner surface or outer surface of the graft layer 100 by a polymer adhesive layer (not shown). Examples of polymer adhesive layers include a silicone based layer and polyurethane based layer.

In yet another configuration (not shown), the bi-iliac module can include two graft layers that are coaxially aligned and fixedly attached to one another by a polymer adhesive layer. The two graft layers can comprise the same fabric or a different fabric. A means for radially supporting a graft layer can be fixedly attached to the inner layer or outer layer of one of the graft layers.

The bi-iliac module 14 can be provided with tapering diameter (not shown) to accommodate the intended iliac or femoral artery sealing location. The first and second ends, 74 and 76, of the bi-iliac module may also include bare stents 104 with hooks 106 to secure the device. The bi-iliac module provides flexibility in sizing the length of the device in-situ, by allowing the first and second ends, 74 and 76, of the bi-iliac module to be implanted where desired for ideal sealing and situating of the mid-portion of the bi-iliac module 14 in the aorta above the aortic bifurcation.

FIGS. 5A-5D illustrate a method of deploying the endoluminal prosthesis to treat an abdominal aortic aneurysm (AAA) that extends from a portion of the aorta caudal the renal arteries (RA) to the aorta iliac junction. The method requires only a single arterial access site.

In the method, it is assumed that the expandable support members and anchoring means of the endoluminal prosthesis are annular stents, formed from shape-memory metal, and that the expandable support members and the anchoring means will radially expand automatically following deployment within the body. From the method described hereinafter, methods employing balloon expansion techniques for introducing endoluminal prosthesis in which the expandable support member and anchoring means do not expand automatically will be readily apparent to one skilled in the art.

Referring to FIGS. 5A-5D, the femoral artery of a leg of the patient to be treated can be accessed percutaneously or by performing an arteriotomy. Using conventional fluoroscopic guidance techniques, a first guide wire can be introduced into the femoral artery. The first guide wire is advanced through the ipsilateral iliac artery, the bi-iliac junction of the aorta, and into the contralateral iliac artery.

Although the ipsilateral iliac artery and the contralateral iliac artery are illustrated as being respectively the right iliac artery (RIA) and left iliac artery (LIA), the ipsilateral iliac artery can be the left iliac artery and the contralateral iliac artery can be the right iliac artery. In this case, the guide wire can then be advanced from the left iliac artery to the right iliac artery.

FIG. 5A shows a first delivery system 200, such as a catheter 202 comprising a nosecone 204 and a cartridge sheath 206, which contains the bi-iliac module 14 in a collapsed condition within the cartridge sheath 204, can be advanced over the guide wire 208 through the ipsilateral iliac artery, the aorta bifurcation (i.e., bi-iliac junction), and into the contralateral iliac artery. Proper placement may be facilitated by use of the radiomarkers (not shown) on the distal end 210 of the cartridge sheath 206.

Once the distal end 210 of the cartridge sheath 206 is positioned just beyond the iliac junction the cartridge sheath 206 can be gradually withdrawn until the bi-iliac module 14 is no longer contained by the cartridge sheath. With the cartridge sheath 206 no longer retaining the bi-iliac module 14 in a collapsed condition, the bi-iliac 14 can radially expand.

FIG. 5B shows that the bi-iliac module 14 can be deployed so that the first end 74 extends into the ipsilateral iliac artery and the second end 76 extends into the contralateral iliac artery. The connection portion 77 of the bi-iliac module is deployed near the apex of bifurcation of the bi-iliac junction of the aorta or directly over the bifurcation so that side opening 78 is aligned with the aorta.

Once the bi-iliac module 14 is deployed across the bi-iliac junction of the aorta, a delivery system 250, such as a catheter 252 comprising a nosecone 254 and a cartridge sheath 256, which contains the aortic module in a collapsed condition within the cartridge sheath, can be used to deploy the aortic module across that abdominal aortic aneurysm within the aorta. FIG. 5C shows that the delivery system 250 containing the aortic module can be advanced over a guide wire 258 through the ipsilateral iliac artery, through the bi-iliac module 14, and into aorta above (i.e., superior) the abdominal aortic aneurysm (i.e., the delivery system is advanced past the renal arteries (RA) within the aorta).

Once a distal end 260 of the cartridge sheath 256 is positioned within the aorta superior the renal arteries, the cartridge sheath 256 can be gradually withdrawn until the proximal end 32 of the aortic module 12 is no longer covered by the cartridge sheath 256. With the cartridge sheath 256 no longer retaining the aortic module 12 in a collapsed condition, the bare stent 44 and the expandable support member 40 of the proximal end 32 will radially expand until bare stent 44 firmly engages the vascular wall of the aorta at the renal junction and the proximal end 32 firmly contacts the wall of the aorta inferior the renal arteries. The proximal end 32 can then be rotated (e.g., about 5 degrees) to embed the radially oriented hooks into the vascular wall of the aorta. Embedding the radially oriented hooks 42 into the vascular wall draws the graft layer 38 of the proximal end 32 into close apposition to the vascular wall so as to form a fluid tight seal between the graft layer 38 of the proximal end 32 and the wall of the aorta.

The cartridge sheath 256 can then be withdrawn over the distal end 34 of the aortic module 12 so that the cartridge sheath 256 no longer retains the connection portion 52 in a collapsed condition. FIG. 5D shows that the support member 62 of the connection portion 52 will radially expand until the outer surface of the connection portion 52 firmly engages the inner surface of the connection portion 77 of the bi-iliac module 14 to form the essentially hourglass shaped junction 16 which interconnects the aortic module 12 and the bi-iliac module 14.

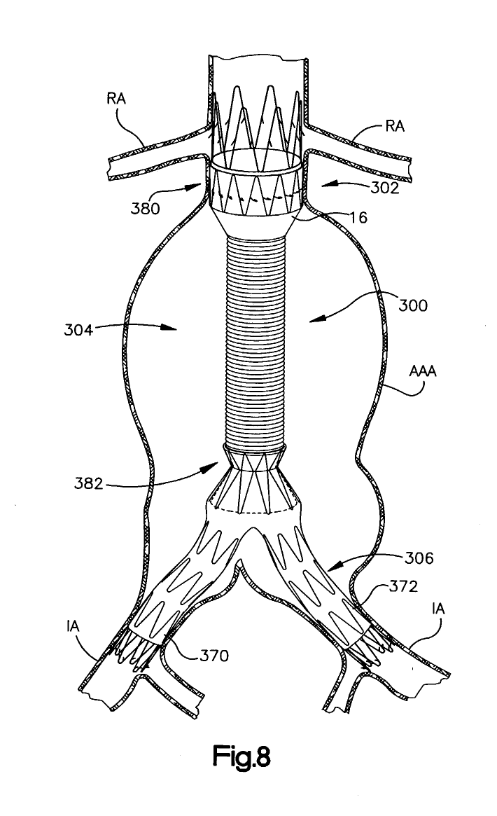

FIGS. 6-8 illustrate a perspective view of an endoluminal prosthesis 300 in accordance with another aspect of the present invention that can be used to treat an abdominal aortic aneurysm that extends from a portion of the aorta caudal the renal arteries to the aorta iliac junction. Referring to FIG. 8, the endoluminal prosthesis 300 can include a proximal sealing collar 302, an aortic main body module 304, and a bi-iliac module 306. Referring to FIG. 6, the proximal sealing collar 302 includes a short-length tubular structure 310 that is radially supported at least a portion of the length of the tubular structure 310. The tubular structure 310 includes an annular first end 312 and a frustoconical second end 314. The annular first end 312 provides a fluid-tight seal between the proximal sealing collar 312 and the aorta in order to create a conduit for blood flow, with full, leak-free exclusion of the aortic aneurysm. The frustoconical second end 314 securely connects with the aortic module 304 and provides a mechanical locking mechanism, which prevents modular disconnection of the frustoconical second end 314 and the aortic module 304.

The tubular structure 310 of the proximal sealing collar 302 includes at least one graft layer 320 and a means for radially supporting the graft layer 322. The graft layer 320 comprises a fabric having sufficient strength to withstand the surgical implantation of the tubular structure 310 and to withstand the blood pressure and other biomechanical forces that are exerted on the structure. The fabric can be formed by weaving or extruding a biocompatible material.

The means 322 for radially supporting the graft layer can comprise a stent 322 that provides lumen patency to proximal seal collar 302. The stent(s) 320 can have a construction similar to any radially expandable stent well-known in the art, which is suitable for vascular implantation. The stent 322 can be fixedly attached to the inner surface or outer surface of the graft layer 320 or integrated into the graft layer 320. The stent 322 can be attached to the inner surface or outer surface of the graft layer 320 by mechanical means. Alternatively, the stent 322 can be attached to the inner surface or outer surface of the graft layer 320 by a polymer adhesive layer (not shown). Examples of polymer adhesive layers include a silicone based layer and polyurethane based layer.

In yet another configuration (not shown), the proximal sealing collar 302 can include two graft layers that are coaxially aligned and fixedly attached to one another by a polymer adhesive layer. The two graft layers can comprise the same fabric or a different fabric. A means for radially supporting a graft layer can be fixedly attached to the inner layer or outer layer of one of the graft layers.

The proximal sealing collar 302 can also include a plurality of substantially radially oriented hooks 324 for ensuring sealing of the proximal sealing collar 302 within the aorta. Preferably, the hooks 324 are curved and extend in an outward direction from the proximal sealing collar 30 such that when the proximal sealing collar 302 is rotated within the aorta, the hooks are deployed, i.e., rotationally embedded, into the aorta, as shown in FIG. 8. Deployment of the radially oriented hooks 324 into the aorta mimics a surgeon's suture and provides secure apposition of the proximal sealing collar 302 to the aorta. The hooks can extend in a substantially coplanar configuration so that the hooks are deployed in an essentially geometric plane, which is substantially perpendicular to the blood flow within the aorta.

FIG. 7 is a perspective view of the aortic module 304 in accordance with a second embodiment of the present invention. The aortic module 304 in accordance with this embodiment comprises a flexible substantially unsupported, and highly conformable tubular structure 330 that connects the bi-iliac module 302 and the proximal sealing collar 302. The aortic module 304 readily conforms to the arterial system acutely as well as accommodates without significant resistance future re-modeling of the arteries, which may occur due to factors such as sac shrinkage and/or arterial disease progression.

The aortic module 304 includes a proximal end 332, a distal end 334, and a main body portion 336 that interconnects the proximal end 332 and the distal end 334. The proximal end 332 and the distal end 334 serve as mechanical junctions, i.e., docking zones for the proximal sealing collar 302 and the bi-iliac module 306, respectively.

The proximal end 332 has a substantially frustoconical shape that is radially supported. The frustoconical shape is used to securely connect of the proximal end 332 of the aortic module 304 within the proximal sealing collar 302 so as to prevent modular disconnection between the proximal sealing collar 302 and the aortic module 304.

The proximal end 332 includes at least one graft layer and a means 340 for radially supporting the graft layer. The construction of the proximal end 332 of the aortic module 304 can be similar to the construction of the proximal sealing collar 302.

An anchoring means 350 can extend from the proximal end 332 of the aortic module 304. The anchoring means 350 can comprise a radially expandable bare stent 352. The bare stent 352 is substantially tubular and can have a construction similar to any vascular stent known in the art.

The bare stent 352 can include axially aligned barbs 354 (or hooks) that extend outwardly from the bare stent 352 and at an angle less 90 degrees with the direction of blood flow through the proximal end 332. When the bare stent 352 is radially expanded, the barbs 354 engage the wall of the aorta and prevent axial migration of the aortic module 304 within the aorta.

The main body portion 336 comprises a highly flexible unsupported tubular graft material. Preferably, the main body portion 336 has corrugated construction formed from radially crimped fabric. The radially crimped fabric includes at least one graft layer. The fabric used to form the one graft layer can be similar to the fabric used to form the proximal sealing collar. Other graft fabrics well known in the art can also be used. The radially crimped fabric can also include additional layers. These additional layers can include other grafts layers and polymer adhesive layers.

The distal end 334 of the aortic module 304 can include a connection portion 360 with a radially supported hourglass configuration. The connection portion can have a construction essentially similar to the construction of the distal end 52 of the aortic module 12 of the endoluminal prosthesis 10. As shown in FIG. 8, the hourglass configuration allows the distal end 334 of the aortic main body module 304 to be connected to the bi-iliac module 306 (FIG. 3) in-situ and form a junction similar to an end-to-side surgical anastomosis.

Referring to FIG. 8, the bi-iliac module 306 of the endoluminal prosthesis 300 can have an essentially similar construction as the bi-iliac module 14 described above and shown in FIG. 3.

The deployment of the endoluminal prosthesis 300 can be achieved in a manner similar to the deployment of the endoluminal prosthesis 10. For example, the bi-iliac module can be deployed over the aortic bifurcation, using a delivery system, so that a first end 370 of the bi-iliac module 306 extends into one iliac artery (IA), a second end 372 of the bi-iliac module extends into the other iliac artery (IA), and a side opening 328 is deployed near the apex of bifurcation or directly into the aorta. The proximal sealing collar 302 can then be deployed using a delivery system in the immediate infrarenal portion of the aorta. The proximal sealing collar module can be sealed to the aorta using the system of rotationally-deployed hooks. Finally, the aortic main body module 306 can be deployed to interconnect the proximal sealing collar module 10 and the bi-iliac module 70, such that the aortic module 304 forms an overlapping junction with the proximal sealing collar 302 and an overlapping end-to-side junction with the bi-iliac module 306.

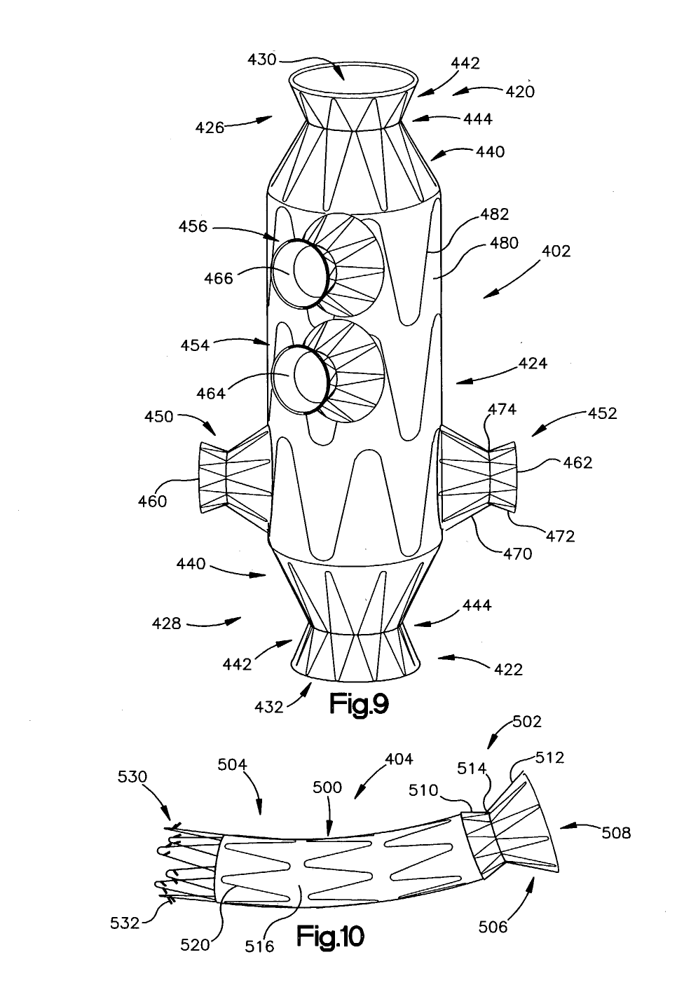

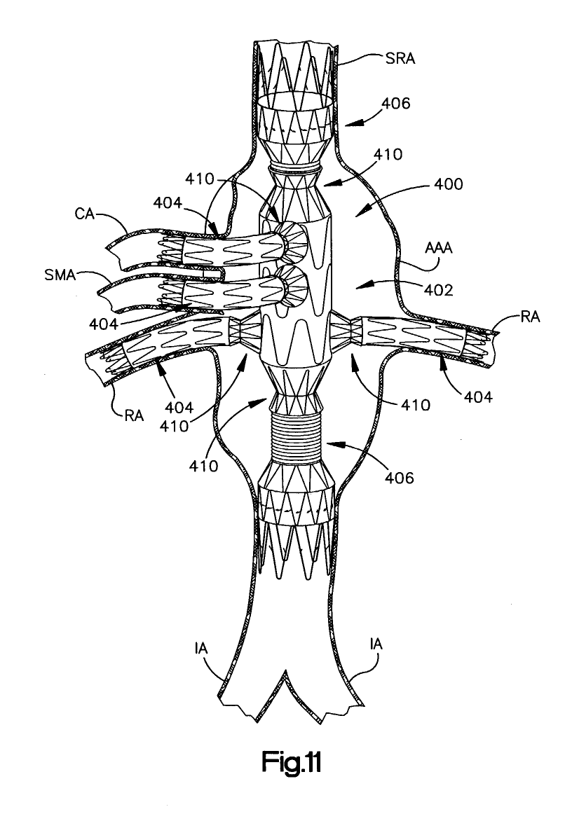

FIGS. 9, 10, and 11 illustrate an endoluminal prosthesis 400 in accordance with yet another aspect of the present invention. The endoluminal prosthesis can be used to treat an aortic abdominal aneurysm that extends across the renal artery junction (i.e., suprarenal abdominal aortic aneurysm). Referring to FIG. 11, the endoluminal prosthesis 400 has a modular design that includes a suprarenal module 402, four branch modules 404, and two aortic modules 406. The aortic modules 404 and branch modules 406 can be connected to the suprarenal module 402 at junctions 410. Each junction 410 can have an essentially hourglass shape and include a converging portion, a diverging portion and a neck portion interconnecting the converging portion and the diverging portion.

Referring to FIG. 9, the suprarenal module 402 includes a flexible tubular segment that includes a first end 420, a second end 422, and a body portion 424 that interconnects the first end 420 and the second end 422. The first end 420 and the second end 422 serve as mechanical junctions for, respectively, the aortic modules 406 (FIG. 11). The first end 420 and the second end 422 are in fluid communication with each other via a lumen (not shown) of the suprarenal module 402.

The first end 420 and the second end 422 comprise, respectively, connection portions 426 and 428. The connection portions 426 and 428 define, respectively, a first opening 430 in the first end 422 and a second opening 432 in the second end 422. The connection portions 426 and 428 each include an annular converging portion 440, an annular diverging portion 442, and an annular neck portion 442 interconnecting the converging portion 440 and the diverging portion 444. The converging portions 440 and the diverging portions 442 taper radially inward to the neck portions 444. The converging portions 440 and diverging portions 442 can have an essentially frustoconical shape that provides the first end 420 and the second end 422 with an essentially hourglass configuration.

The body portion 424 includes a first renal connection portion 450, a second renal connection portion 452, a superior mesenteric connection portion 454, and a celiac connection portion 456 that define, respectively, side openings 460, 462, 464, and 466 in the body portion 424. The side openings 460, 462, 464, and 466 are in fluid communication with the lumen of the suprarenal module 402, such that fluid will flow from the lumen and out of the side openings 460, 462, 464, and 466. As may be seen in FIG. 11, the connection portions 450, 452, 454, and 456 can be located on body portion 424 such that when the suprarenal module 402 is deployed in the aorta the connection portions can be aligned respectively with the renal arteries (RA), the superior mesenteric artery (SMA), and the celiac artery (CA).

Each connection portion (e.g., 452) can include an annular converging portion 470, an annular diverging portion 472, and a neck portion 474 that interconnects the annular converging portion 470 and the annular diverging portion 472. The converging portion 470 and the diverging portions 472 can taper radially inward to the neck portions 474. The diverging portions 474 can be essentially frustoconical in configuration and perpendicularly offset from the lumen to provide each connection portion 450, 452, 454, and 456 with an essentially hourglass configuration. FIG. 11 shows that the essentially hourglass connection portions 450, 452, 454, and 456 of the suprarenal module 402 allow the branch modules 404 to be connected to the suprarenal module 402 with a mechanical locking fit.

The suprarenal module 402 can be formed from at least one graft layer 480 and a means 482 for radially supporting the graft layer 480. The graft layer 480 can comprise a fabric having sufficient strength to withstand the surgical implantation of the suprarenal module 402 and to withstand the blood pressure and other biomechanical forces that are exerted on the structure. The fabric can be formed by weaving or extruding a biocompatible material.

The means 482 for radially supporting the graft layer 480 can comprise at least one stent 482 that provides lumen patency to suprarenal module 402. The stent(s) 482 can have a construction similar to any radially expandable stent well-known in the art, which is suitable for vascular implantation. The stent 482 can be fixedly attached to the inner surface or outer surface of the graft layer 480 or integrated into the graft layer 480. The stent 482 can be attached to the inner surface or outer surface of the graft layer 480 by mechanical means. Alternatively, the stent 482 can be attached to the inner surface or outer surface of the graft layer 480 by a polymer adhesive layer (not shown). Examples of polymer adhesive layers include a silicone based layer and polyurethane based layer.

In yet another configuration (not shown), the suprarenal module 402 can include two graft layers that are coaxially aligned and fixedly attached to one another by a polymer adhesive layer. The two graft layers can comprise the same fabric or a different fabric. A means for radially supporting a graft layer can be fixedly attached to the inner layer or outer layer of one of the graft layers.

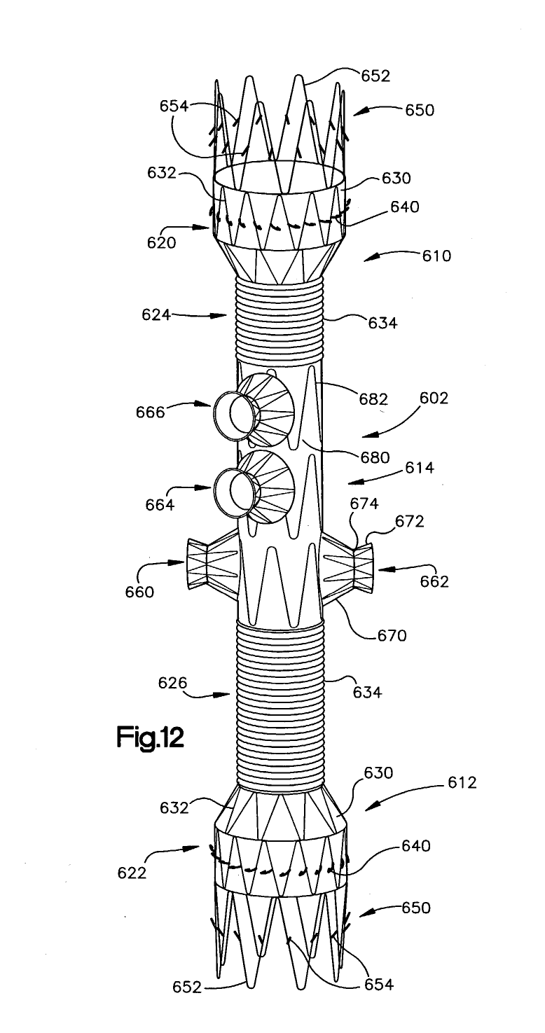

Referring to FIG. 10, the branch modules 404 are connected respectively to the first renal connection portion 450, the second renal connection portion 452, the superior mesenteric connection portion 454, and the celiac portion 456. The branch modules 404 interconnect the suprarenal module 402 with branch arteries of the aorta (i.e., the renal arteries, the superior mesenteric artery, and the celiac artery). Although the branch modules 404 are illustrated as having similar lengths and diameters, the lengths and diameters of the branch modules 404 can vary depending on the distance from the connection portions 450, 452, 454, and 456 to the specific artery, which the branch module 404 connects, and the diameter of the specific branch artery.

FIG. 10 illustrates an exemplary embodiment of a branch module 404. The branch modules 404 all have a similar construction. Accordingly the construction of only one of the branch modules 404 will be discussed below.

The branch module 404 comprises a flexible hollow tubular segment 500 that includes a first end 502 and an second end 504. The first end 502 and the second end 504 are in fluid communication with each other by a main lumen (not shown) of the branch module 404.

The first end 502 includes a connection portion 506 that defines an opening 508 in the first end 502. The connection portion 506 can include an annular converging portion 510, an annular diverging portion 512, and an annular neck portion 514 that interconnects the converging portion 510 and the diverging portion 512. The converging portion 510 and the diverging portion 512 taper radially inward to the neck portion 514. The converging portion 510 and the diverging portion 512 can be essentially frustoconical (i.e., funnel shaped) in configuration to provide the connection portion 506 with an essentially hourglass configuration. As shown in FIG. 11, the, hourglass connection portion 512 of branch module 404 allows the first end 502 of the branch module 404 to be connected to the connection portions of the suprarenal module 402 with a mechanical locking fit.

The branch module 404 can be formed from at least one graft layer 516 and a means 520 for radially supporting the graft layer 516. The graft layer 516 can comprise a fabric having sufficient strength to withstand the surgical implantation of the branch module 404 and to withstand the blood pressure and other biomechanical forces that are exerted on the branch module 404. The fabric can be formed by weaving or extruding a biocompatible material.

The means 520 for radially supporting the graft layer 516 can comprise at least one stent 520 that provides lumen patency to the branch module 404. The stent(s) 520 can have a construction similar to any radially expandable stent 520 well-known in the art, which is suitable for vascular implantation. The stent 520 can be fixedly attached to the inner surface or outer surface of the graft layer 516 or integrated into the graft layer 520. The stent 520 can be attached to the inner surface or outer surface of the graft layer 520 by mechanical means. Alternatively, the stent 520 can be attached to the inner surface or outer surface of the graft layer 516 by a polymer adhesive layer (not shown). Examples of polymer adhesive layers include a silicone based layer and polyurethane based layer.

In yet another configuration (not shown), the branch module 404 can include two graft layers that are coaxially aligned and fixedly attached to one another by a polymer adhesive layer. The two graft layers can comprise the same fabric or a different fabric. A means for radially supporting a graft layer can be fixedly attached to the inner layer or outer layer of one of the graft layers.

Optionally, the second end 504 of the branch module 404 can be provided with tapering diameter (not shown) to accommodate the intended branch artery sealing location. The second end 504 of the branch module can also include a bare stent 430 with hooks 432 (or barbs) to secure the branch module 404 within the vasculature.

The aortic modules 406 of the endoluminal prosthesis 400 can have an essentially similar construction as the aortic module 12 described above and shown in FIG. 2. The lengths and diameters of the aortic modules 406, however, can vary depending on the distance from the connection portions the length and diameter of the aneurysm that extend across the renal artery junction.

The endoluminal prosthesis 400 can be deployed by implanting the aortic module 406 in a suprarenal portion of the aorta (e.g., using a delivery system, such as a catheter with a nosecone and a cartridge sheath). Following implantation of the aortic module 406, the suprarenal module 406 can be deployed (e.g., using a delivery system) across the abdominal aortic aneurysm (AAA). The suprarenal module 402 can be connected to the suprarenal aortic module 406 with a mechanical locking fit. A second aortic module 406 can then be deployed (e.g., using a delivery system) caudal the abdominal aortic aneurysm. The second aortic module can be connected to the suprarenal module 402 with a mechanical locking fit. The branch modules 404 can then be individually deployed (e.g., using a delivery system) through the suprarenal module 402 and to the branch arteries (RA), (SMA), and (CA). The branch modules 404 can be connected to the suprarenal module 402 with a mechanical locking fit. It will be appreciated by one skilled in the art based on the methods described above with respect to deployment of the endoluminal prosthesis 10, that the endoluminal prosthesis 400, like the endoluminal prosthesis 10, can be deployed using only unilateral arterial access.

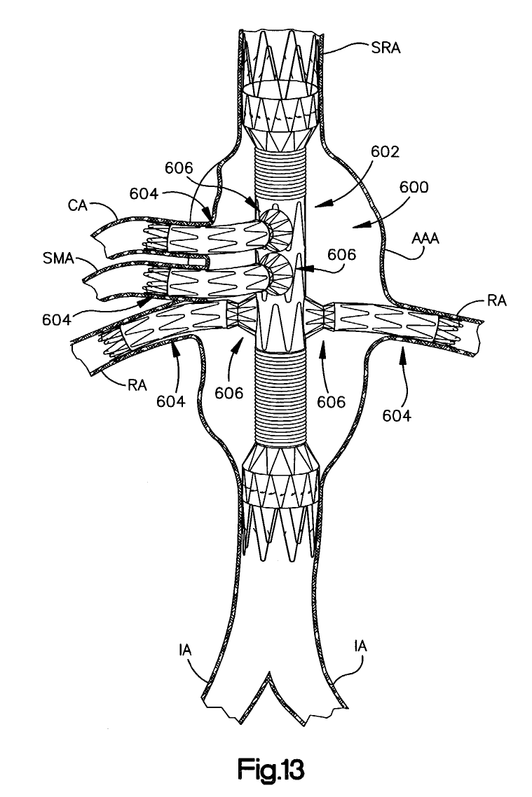

FIGS. 12 and 13 illustrate an endoluminal prosthesis 600 in accordance with yet another aspect of the present invention. The endoluminal prosthesis 600 can be used to treat an aortic abdominal aneurysm that extends across the renal artery junction. Referring to FIG. 13, the endoluminal prosthesis 600 has a modular design that includes a suprarenal module 602 and four branch modules 604. The branch modules 604 can be connected to the suprarenal module 602 at junctions 606. Each junction 606 can have an essentially hourglass shape and include a converging portion, a diverging portion and a neck portion interconnecting the converging portion and the diverging portion.

Referring to FIG. 12, the suprarenal module 602 includes a first end 610, a second end 612, and a body portion 614 that interconnects the first end 610 and the second end 612. The first end 610 and the second end 612 are in fluid communication with each other via the lumen (not shown) of the suprarenal module 602.

The first end 610 and the second end 612 include substantially frustoconical portions 620 and 622, which are radially supported at least a portion of the length of the frustoconical portions 620 and 622 and highly flexible unsupported tubular portions 624 and 626. The first end 610 and the second end 612 provide a fluid-tight seal with the aorta and create a conduit for blood flow, with full, leak-free exclusion of the aortic aneurysm.

The frustoconical portions 620 and 622 of the first end 610 and the second end 612 can be formed from at least one graft layer 630 and a means 632 for radially supporting the graft layer 630. The graft layer 630 can comprise a fabric having sufficient strength to withstand the surgical implantation of the suprarenal module 602 and to withstand the blood pressure and other biomechanical forces that are exerted on the suprarenal module 602. The fabric can be formed by weaving or extruding a biocompatible material.

The means 632 for radially supporting the graft layer 630 can comprise at least one stent 632 that provides lumen patency to the suprarenal module 602. The stent(s) 632 can have a construction similar to any radially expandable stent 632 well-known in the art, which is suitable for vascular implantation. The stent 632 can be fixedly attached to the inner surface or outer surface of the graft layer 630 or integrated into the graft layer 630. The stent 632 can be attached to the inner surface or outer surface of the graft layer 630 by mechanical means. Alternatively, the stent 632 can be attached to the inner surface or outer surface of the graft layer 630 by a polymer adhesive layer (not shown). Examples of polymer adhesive layers include a silicone based layer and polyurethane based layer.

In yet another configuration (not shown), the frustoconical portions 620 and 622 can include two graft layers that are coaxially aligned and fixedly attached to one another by a polymer adhesive layer. The two graft layers can comprise the same fabric or a different fabric. A means for radially supporting a graft layer can be fixedly attached to the inner layer or outer layer of one of the graft layers.

The frustoconical portions 620 and 622 of the first end 610 and the second end 612 can each include pluralities of substantially radially oriented hooks 640 for ensuring sealing of the first end 610 and the second end 612 within the aorta. Preferably, the hooks 640 are curved and extend in an outward direction from the first end 610 and the second end 612 such that when the first end 610 and the second end 612 are rotated within the aorta, the hooks are deployed, i.e., rotationally embedded, into the wall of aorta, as shown in FIG. 13. Deployment of the radially oriented hooks 640 into the aorta mimics a surgeon's suture and provides secure apposition of the first end 610 and the second en 612 to the aorta. The hooks 640 can extend in a substantially coplanar configuration that is essentially perpendicular to the axially extending proximal end and to the blood flow through the proximal end of the aortic module. The hooks 640 can be deployed in an essentially geometric plane, which is substantially perpendicular to the blood flow within the aorta.

The tubular portions 624 and 626 can have a corrugated construction formed from radially crimped fabric. The radially crimped fabric includes at least one graft layer 634. The fabric used to form the one graft layer can be similar to the fabric used to form proximal end. Other graft fabrics well known in the art can also be used. The radially crimped fabric can also include additional layers. These additional layers can include other grafts layers and polymer adhesive layers.

Anchoring means 650 can extend from the first end 610 and the second end 612 of the suprarenal module 602. The anchoring means 650 can comprise radially expandable bare stents 652. The bare stents 652 can be substantially tubular and can have a construction similar to any vascular stent known in the art.

The bare stents 652 can includes axially aligned barbs 654 (or hooks) that extend outwardly from the bare stent 652 and at an angle less 90 degrees with the direction of blood flow through the suprarenal module 602. When the bare stent 652 is radially expanded, the barbs 654 engage the wall of the aorta and prevent migration of the suprarenal module 602 within the aorta.

The body portion 614 includes a first renal connection portion 660, a second renal connection portion 662, a superior mesenteric connection portion 664, and a celiac connection portion 666, which each define side openings in the body portion. The side openings are in fluid communication with the lumen, such that fluid will flow from the lumen and out of the side openings. The connection portions 660, 662, 664, and 666 can be located on the body portion 614 such that when the suprarenal module 602 is deployed in the aorta the connection portions 660, 662, 664, and 666 can be aligned respectively with the renal arteries, the superior mesenteric artery, and the celiac artery.

Each connection portion (e.g., 662) can include an annular converging portion 670, an annular diverging portion 672, and a neck portion 674 that interconnects the annular converging portion 670 and the annular diverging portion 672. The converging portions 670 and the diverging portions 672 can taper radially inward to the neck portions 674. The converging portions 670 and the diverging portions 672 can be essentially frustoconical in configuration and perpendicularly offset from the lumen to provide each connection portion 660, 662, 664, and 666 with an essentially hourglass configuration. FIG. 13 shows that the essentially hourglass connection portions 660, 662, 664, and 666 of the suprarenal module 602 allow the branch modules to be connected to the suprarenal module with a mechanical locking fit.

The body portion 614 of the suprarenal module 602 can be formed from at least one graft layer 680 and a means 682 for radially supporting the graft layer 680. The graft layer 680 can comprise a fabric having sufficient strength to withstand the surgical implantation of the suprarenal module 602 and to withstand the blood pressure and other biomechanical forces that are exerted on the structure. The fabric can be formed by weaving or extruding a biocompatible material.

The means 682 for radially supporting the graft layer 680 can comprise at least one stent 682 that provides lumen patency to the body portion 614 of the suprarenal module 602. The stent(s) 682 can have a construction similar to any radially expandable stent well-known in the art and which is suitable for vascular implantation. The stent 682 can be fixedly attached to the inner surface or outer surface of the graft layer 680 or integrated into the graft layer 680. The stent 682 can be attached to the inner surface or outer surface of the graft layer 680 by mechanical means. Alternatively, the stent 682 can be attached to the inner surface or outer surface of the graft layer 680 by a polymer adhesive layer (not shown). Examples of polymer adhesive layers include a silicone based layer and polyurethane based layer.

In yet another configuration (not shown), the body portion 614 of the suprarenal module 602 can include two graft layers that are coaxially aligned and fixedly attached to one another by a polymer adhesive layer. The two graft layers can comprise the same fabric or a different fabric. A means for radially supporting the graft layer can be fixedly attached to the inner layer or outer layer of one of the graft layers.

Referring to FIG. 13, the branch modules are connected respectively to the first renal connection portion 660, the second renal connection portion 662, the superior mesenteric connection portion 664, and the celiac portion 666. The branch modules 604 interconnect the suprarenal module 602 with branch arteries of the aorta (i.e., the renal arteries, the superior mesenteric artery, and the celiac artery).

The branch modules 604 of the endoluminal prosthesis 600 can have an essentially similar construction as the branch modules 404 described above and shown in FIG. 10. Although the branch modules 604 are illustrated as having similar lengths and diameters, the lengths and diameters of the branch modules 604 can vary depending on the distance from the connection portions 660, 662, 664, and 666 to the specific artery, which the branch module 604 connects, and the diameter of the specific branch artery.

The endoluminal prosthesis 600 can be deployed by implanting the suprarenal module 602 across the abdominal aortic aneurysm (AAA) (e.g., using a second delivery system, such as a catheter with a nosecone and a cartridge sheath). The branch modules 604 can then be individually deployed (e.g., using a delivery system) through the suprarenal module 602 and to the branch arteries (RA), (SMA), and (CA). The branch modules 604 can be connected to the suprarenal module 602 with a mechanical locking fit to form junctions 606. It will be appreciated by one skilled in the art based on the methods described above with respect to deployment of the endoluminal prosthesis 10, that the endoluminal prosthesis 600, like the endoluminal prosthesis 10, can be deployed using only unilateral arterial access.

From the above description of the invention, those skilled in the art will perceive improvements, changes and modification. Such improvements, changes and modifications within the skill of the art are intended to be covered by the appended claims. For example, the hooks of the proximal sealing collar, the aortic module, the bi-iliac module, the suprarenal module, and/or the branch modules can have a barbed end configuration similar to a fishhook to prevent dislodgement from the artery wall. The barbed end preferably employs a rough-textured surface to promote a heightened localized response and increased scar tissue formation. Heightened localized response and increased scar tissue formation further enhances the fixation of the hooks within the wall of the aorta. The rough textured surface on the barbed hooks can be provided by various methods. Examples of methods that can be used to provide a rough textured surface on a hook include selective metallic coating of a metallic hook, micro-bead blasting a hook, injection molding a hook from a polymer material with the desired roughness, and forming a hook of multiple materials and dissolving away one or more of the materials.

In yet another aspect of the present invention, at least one of the modules can have varying biological, physical, and/or chemical properties associated with the inner and/or outer surface of the module such that the inner surface of the module is optimized to reduce biological responses and/or the outer surface is optimized to promote biological responses. Examples of variations in the physical properties include the inner surface of at least one module being smooth to lessen clotting or other solid particle deposition and/or the outer surface of at least one module being rough to increase the surface area for foreign material and increase biologic host response. Examples of variations in the chemical properties include the inner surface of at least one module incorporating an anti-thrombogenic agent, such as heparin, to decrease the propensity for clot formation and/or the outer surface incorporating a thrombogenic agent, such as thrombin, to increase the propensity for clot formation.

Having described the invention, the following is claimed:

1. An endoluminal prosthesis comprising:
a radially expandable tubular segment having a first end, a second end, a lumen interconnecting the first end and the second end, and a connection portion defining an opening in the tubular segment in fluid communication with the lumen, the connection portion including a converging portion, an annular diverging portion and an annular neck portion interconnecting the converging portion and the diverging portion.

2. The endoluminal prosthesis of claim 1, wherein the diverging portion comprises an annular member that tapers radially inward to the neck portion.

3. The endoluminal prosthesis of claim 2, wherein the diverging portion is provided in an open configuration by a support member.

4. The endoluminal prosthesis of claim 1 wherein the connection portion has an essentially hourglass shape.

5. The endoluminal prosthesis of claim 1, wherein the first end includes the opening defined by the connection portion and the second end includes a second opening, the opening defined by the connection portion and the second opening being in fluid communication with each other via the lumen.

6. The endoluminal prosthesis of claim 1 being deployed within the vasculature to treat a infrarenal abdominal aortic aneurysm.

7. The endoluminal prosthesis of claim 1 being deployed within the vasculature to treat a suprarenal abdominal aortic aneurysm.

8. The endoluminal prosthesis of claim 5, further comprising a second radially expandable tubular segment, the second segment having a first end, a second end, a lumen interconnecting the first end and the second end, and a second connection portion defining an opening in a mid-portion of the second tubular segment between the first end and the second end of the second segment, the opening in the mid-portion being in fluid communication with the lumen of the second segment, the second connection portion being capable of joining with the first connection portion in situ to form a mechanical junction that allows fluid flow between the first segment and the second segment.

9. The endovascular prosthesis of claim 8, wherein the second connection portion includes a converging portion, an annular diverging portion and an annular neck portion interconnecting the converging portion and the diverging portion.

10. The endoluminal prosthesis of claim 9, wherein the diverging portion of the second segment is perpendicularly offset from the lumen of the second segment to provide the second connection portion with an essentially hourglass shape.

11. The endoluminal prosthesis of claim 9, wherein the first connection portion includes an outer surface and the second connection portion includes an inner surface, the inner surface of the second connection portion engaging the outer surface of the first connection portion when the first connection portion and second connection portion are joined to form the mechanical junction.

12. The endoluminal prosthesis of claim 1, wherein the first segment includes a means for creating a fluid tight seal between the first segment and a wall of a body lumen.

13. The endoluminal prosthesis of claim 12, wherein the means for creating a fluid-tight seal comprises a plurality of substantially radially oriented hooks that extend from the first segment and enter the wall of the lumen in a rotational manner to draw the first segment into close apposition with the wall.

14. The endoluminal prosthesis of claim 13, wherein the first segment includes an anchoring means to inhibit axial motion of the first segment within the lumen.

15. An endoluminal prosthesis comprising:
a first radially expandable tubular segment that includes a first lumen;
a second radially expandable tubular segment that includes a second lumen; and
a junction connecting the first radially expandable tubular segment and the second radially expandable tubular segment, the junction allowing fluid flow from the first lumen to the second lumen, the junction comprising an annular converging portion, an annular diverging portion and an annular neck portion interconnecting the converging portion and the diverging portion.

16. The endoluminal prosthesis of claim 15 wherein the first segment includes a first connection portion and the second segment includes a second connection portion, the first connection portion engaging the second connection portion to form the junction.

17. The endoluminal prosthesis of claim 16 wherein the first connection portion includes a first converging portion, a first annular diverging portion and a first annular neck portion interconnecting the first converging portion and the first diverging portion and the second connection portion includes a second converging portion, a second annular diverging portion and a second annular neck portion interconnecting the second converging portion and the second diverging portion.

18. The endoluminal prosthesis of claim 17, wherein the first diverging portion includes a first support member that provides the diverging portion in an open configuration and the second diverging portion includes a second support member that provides the second diverging portion in an open configuration.

19. The endoluminal prosthesis of claim 18, wherein the first segment includes a first end and a second end, the first connection portion providing an opening in the first end which is in fluid communication with the first lumen.

20. The endoluminal prosthesis of claim 19, wherein the first diverging portion and first converging portion are tapered radially inward to the first neck portion.

21. The endoluminal prosthesis of claim 20 being deployed within the vasculature to treat a suprarenal abdominal aortic aneurysm.

22. The endoluminal prosthesis of claim 18, wherein the second segment includes a first end, a second end in fluid communication with the first end via the second lumen, and a mid-portion between the first end and the second end, the second connection member defining a side opening in the mid-portion.

23. The endoluminal prosthesis of claim 22, wherein the second diverging portion of the second connection portion comprises an annular member that is tapered radially inward to the second lumen.

24. The endoluminal prosthesis of claim 23 being deployed within the vasculature to treat an infrarenal abdominal aortic aneurysm

25. The endoluminal prosthesis of claim 15, wherein the first segment includes a means for creating a fluid tight seal between the segment and a wall of a body lumen.

26. The endoluminal prosthesis of claim 25, wherein the means for creating a fluid-tight seal comprises a plurality of substantially radially oriented hooks that extend from the first segment and enter the wall of the lumen in a rotational manner to draw the first segment into close apposition to the wall.

27. The endoluminal prosthesis of claim 25, wherein the first segment includes an anchoring means to inhibit axial motion of the first segment within the lumen.

28. An endoluminal prosthesis for treating a suprarenal abdominal aortic aneurysm, the endoluminal prosthesis comprising:
a radially expandable tubular trunk segment having a first end, a second end, a lumen interconnecting the first end and the second end, and at least two connection portions defining openings in a mid-portion of the trunk segment between the first end and the second end of the trunk segment, the openings in the mid-portion being in fluid communication with the lumen of the trunk segment, at least one of the connection portions comprising an annular converging portion, an annular diverging portion and an annular neck portion interconnecting the converging portion and the diverging portion.

29. The endoluminal prosthesis of claim 28, further comprising a radially expandable tubular branch segment, the branch segment having a first end, a second end, a lumen interconnecting the first end and the second end, and a second connection portion, the second connection portion being capable of joining with at least one of the connection portions of the trunk segment in situ to form a mechanical junction that allows fluid flow between the trunk segment and the branch segment.

30. The endoluminal prosthesis of claim 29, wherein the second connection portion includes a converging portion, an annular diverging portion and an annular neck portion interconnecting the converging portion and the diverging portion.

31. The endoluminal prosthesis of claim 30, wherein the first end of the branch segment includes an opening defined by the second connection portion and the second end includes a means for attaching the second end of the branch segment within a branch artery of the aorta.

32. The endoluminal prosthesis of claim 31, wherein the branch artery comprises a renal artery, a superior mesenteric artery, or a celiac artery.

33. The endoluminal prosthesis of claim 28, wherein the trunk segment includes four connection portions defining four openings in the mid-portion of the trunk segment and at least one of the four connection portions comprises an annular converging portion, an annular diverging portion, and an annular neck portion interconnecting the converging portion and the diverging portion.

34. The endoluminal prosthesis of claim 33, wherein the four connection portions of the trunk segment each include an annular converging portion, an annular diverging portion and an annular neck portion interconnecting the converging portion and the diverging portion.

35. The endoluminal prosthesis of claim 34, further comprising four radially expandable branch segments, each branch segment having a first end, a second end, a lumen interconnecting the first end and the second end, and a second connection portion, the second connection portion of at least one of the branch segments being capable of joining with at least one of the connection portions of the trunk segment in situ to form a mechanical junction that allows fluid flow between the trunk segment and the branch segment.

36. A method of treating an aortic aneurysm, said method comprising the steps of:
deploying a first radially expandable tubular segment, the first segment having a first end, a second end, a lumen interconnecting the first end and the second end, and a first connection portion defining an opening in a mid-portion of the first segment between the first end and the second end, the first connection portion including a converging portion, an annular diverging portion and an annular neck portion interconnecting the converging portion and the diverging portion; and
deploying a second radially expandable tubular segment, the second segment including a distal end, a proximal end, a lumen interconnecting the distal end and the proximal end, and a second connection portion defining an opening in the either end in fluid communication with the lumen, the second connection portion including a converging portion, an annular diverging portion and an annular neck portion interconnecting the converging portion and the diverging portion, the second connection portion and the first connection portion forming an end-to-side junction which allows fluid flow between the first segment and the second segment.

37. A method of treating an infrarenal abdominal aortic aneurysm requiring only unilateral arterial access, said method comprising the steps of:
advancing a guide wire through an arterial access site in the ipsilateral iliac or femoral artery, over the aortic bifurcation and at least partially into the contralateral iliac artery,
advancing over the guide wire a first delivery system containing a first radially expandable tubular segment, the first segment having a first end, a second end, a lumen interconnecting the first end and the second end, and a first connection portion defining an opening in a mid-portion of the first segment between the first end and the second end, the first connection portion including a converging portion, an annular diverging portion and an annular neck portion interconnecting the converging portion and the diverging portion,
deploying the first segment into both iliac arteries, over the aortic bifurcation, such that said the opening is deployed near the apex of the bifurcation or directly into the aorta,
re-positioning the guide wire, or placing a new wire, so that it extends from the arterial access site through the opening in the mid-portion of the first segment and into the aorta,
advancing over the guide wire placed into the aorta a second delivery system containing a second radially expandable tubular segment, the second segment including a distal end, a proximal end, a lumen interconnecting the distal end and the proximal end, and a second connection portion defining an opening in the distal end in fluid communication with the lumen, the second connection portion including a converging portion, an annular diverging portion and an annular neck portion interconnecting the converging portion and the diverging portion, and
deploying the second segment into the aorta such that the second connection portion and the first connection portion form and end-to-side junction that allows fluid flow between the first segment and the second segment.

38. A method of treating an suprarenal abdominal aortic aneurysm requiring only unilateral arterial access, said method comprising the steps of:
deploying a radially expandable tubular trunk segment having a first end, a second end, a lumen interconnecting the first end and the second end, and at least two connection portions defining openings in a mid-portion of the trunk segment between the first end and the second end of the trunk segment, the openings in the mid-portion being in fluid communication with the lumen of the trunk segment, at least one of the connection portions comprising an annular converging portion, an annular diverging portion and an annular neck portion interconnecting the converging portion and the diverging portion; and
deploying a radially expandable tubular branch segment, the branch segment having a first end, a second end, a lumen interconnecting the first end and the second end, and a second connection portion, the second connection portion including a converging portion, an annular diverging portion and an annular neck portion interconnecting the converging portion and the diverging portion, the second connection portion being capable of joining with at least one of the connection portions of the trunk segment in situ to form a mechanical junction that allows fluid flow between the trunk segment and the branch segment.

39. An endovascular prosthesis comprising,
a radially expandable tubular graft layer having a first end, a second end and a lumen extending between the first end and the second end, the first end including a plurality of substantially radially oriented hooks that extend from the graft layer to provide a fluid tight seal between graft layer of the first end and a wall of the vasculature.

40. The endovascular prosthesis of claim 39, wherein the hooks enter the wall of the vasculature in a rotational manner to draw the first end into close apposition to the wall.

41. The endovascular prosthesis of claim 40, wherein the hooks extend in a substantially coplanar configuration that is essentially perpendicular to blood flow through the endovascular prosthesis.

42. The endovascular prosthesis of claim 41, wherein the hooks are deployed in an essentially geometric plane, that is essentially perpendicular to the blood flow within the vasculature.

43. The endovascular prosthesis of claim 39, wherein the hooks include a rough-textured surface to promote a heightened localized biological response.

44. The endovascular prosthesis of claim 39, further comprising an anchoring means for securing the endovascular prosthesis within the vasculature.

45. The endovascular prosthesis of claim 44, wherein the anchoring means secures the endovascular prosthesis within the vasculature by substantially inhibiting axial motion of the endovascular prosthesis relative to the vasculature.

46. The endovascular prosthesis of claim 39 being deployed to treat at least one of a suprarenal abdominal aortic aneurysm and an infrarenal abdominal aortic aneurysm.

47. An endovascular prosthesis comprising,
a radially expandable tubular graft layer having a first end, a second end and a lumen extending along an axis between the first end and the second end, the first end including a plurality of substantially radially oriented hooks, the hooks being curved to enter a wall of a vasculature upon axial rotation of the endovascular prosthesis and draw the first end into close apposition to the wall so as to form a fluid tight seal between the graft layer of the first end and the wall of the vasculature

48. The endovascular prosthesis of claim 47, wherein the hooks extend in a substantially coplanar configuration that is essentially perpendicular to the axis.

49. The endovascular prosthesis of claim 48, wherein the hooks include a rough-textured surface to promote a heightened localized biological response, increase scar tissue formation, and enhance the fixation of the hooks.

50. The endovascular prosthesis of claim 49, further comprising an anchoring means for substantially inhibiting axial motion of the endovascular prosthesis relative to the vasculature.

51. The endovascular prosthesis of claim 50, wherein the anchoring means includes hooks which penetrate the vasculature, the hooks of the anchoring means being deployed at an angle of less than 90 degrees with the direction of blood flow through the endovascular prosthesis.

52. The endovascular prosthesis of claim 47 being deployed to treat at least one of a suprarenal abdominal aortic aneurysm and an infrarenal abdominal aortic aneurysm.

53. An endovascular prosthesis comprising,
a radially expandable tubular graft layer having a first end, a second end and a lumen extending along an axis between the first end and a second end, the first end including an anchoring means for substantially inhibiting axial motion of the endovascular prosthesis relative to the vasculature and a sealing means to provide a fluid tight seal between graft layer of the first end and a wall of the vasculature, the anchoring means and sealing means being separate from one another.

54. The endovascular prosthesis of claim 53, wherein the sealing means comprises a plurality of substantially radially oriented hooks, the hooks being curved to enter a wall of a vasculature upon axial rotation of the endovascular prosthesis and draw the first end into close apposition to the wall so as to form a fluid tight seal between the graft layer of the first end and the wall of the vasculature.

55. The endovascular prosthesis of claim 54, wherein the hooks extend in a substantially coplanar configuration that is essentially perpendicular to blood flow through the endovascular prosthesis.

56. A method of deploying an endovascular prosthesis within a vasculature, the method comprising the steps of:
providing an endovascular prosthesis that includes a radially expandable tubular graft layer having a first end, a second end, a lumen extending between the first end and the second end, and a plurality of substantially radially oriented hooks extending from the first end, the hooks being curved to enter a wall of the vasculature upon axial rotation of the endovascular prosthesis, and
rotationally embedding the substantially radially oriented hooks of the first end into the wall of the vasculature to achieve a fluid-tight seal.

57. A method of forming a fluid tight seal between an endovascular prosthesis and a wall of a vasculature, the method comprising the steps of:
providing an endovascular prosthesis that includes a radially expandable tubular graft layer having a first end, a second end, a lumen extending between the first end and the second end, and a plurality of substantially radially oriented hooks extending from the first end, the hooks being curved to enter a wall of the vasculature upon axial rotation of the endovascular prosthesis, and
rotationally embedding the substantially radially oriented hooks of the first end into the wall of the vasculature.
